(19) United States
(12) Patent Application Publication
(10) Pub. No.: US 2019/0117694 A1
(43) Pub. Date: Apr. 25, 2019

(54) METHODS OF PREVENTING PLATELET ALLOIMMUNIZATION AND ALLOIMMUNE PLATELET REFRACTORINESS AND INDUCTION OF TOLERANCE IN TRANSFUSED RECIPIENTS

(71) Applicant: BLOODWORKS, Seattle, WA (US)

(72) Inventor: Sherrill J. Slichter, Vashon, WA (US)

(21) Appl. No.: 16/134,773

(22) Filed: Sep. 18, 2018

Related U.S. Application Data
(63) Continuation of application No. 13/204,332, filed on Aug. 5, 2011, now abandoned.
(60) Provisional application No. 61/371,491, filed on Aug. 6, 2010.

(51) Int. Cl. A61K 35/19; A61K 41/00

(52) U.S. Cl. CPC A61K 35/19; A61K 41/0023

(57) ABSTRACT

Methods and compositions for the prevention or reduction of platelet transfusion associated complications are provided. Methods are provided to modify donor platelets prior to transfusion to prevent or reduce Alloimmune platelet refractoriness.

### CROSS REFERENCE TO RELATED APPLICATIONS

This application is a continuation of U.S. patent application Ser. No. 13/204,332, filed on Aug. 5, 2011, which claims the benefit of U.S. Provisional Application No. 61/371,491, filed Aug. 6, 2010, each of which is hereby incorporated by reference in its entirety.

### STATEMENT OF GOVERNMENT SUPPORT

This invention was made with United States Government support under U.S. Army Medical Research and Materiel Command Grant No. 07328001. The United States Government has certain rights in this invention.

### FIELD

This invention is directed to methods of preventing transfusion related complications in recipients of donor blood or components thereof.

### BACKGROUND

Blood transfusion is the process of receiving blood products into one's circulation intravenously. Transfusions are used in a variety of medical conditions to replace lost components of the blood. Early transfusions used whole blood, but modern medical practice commonly uses only components of the blood, such as red blood cells, white blood cells, plasma, clotting factors, and platelets.

Transfusions of blood products is associated with complications, including immunologic transfusion reactions. One example of such an immunologic response is alloimmunization, an immune response generated in an individual or strain of one species in response to an alloantigen from a different individual or strain of the same species. Alloimmunization can result in the rejection of transfused or transplanted tissues, such as platelets, which leads to platelet refractoriness.

As a consequence, the platelet donor and recipient must be closely matched to avoid this immunological reaction. This process of matching can be a complicated and difficult procedure due to the complexity of the marker system that determines compatibility. Thus, the problem of alloimmunization of recipients against donor blood products is a major problem in transfusion medicine. The present invention provides solutions to these and other unmet needs in transfusion medicine.

### SUMMARY

Described herein are methods and compositions for the prevention or reduction of alloimmune platelet refractoriness prior to transfusion by modifying donor platelets.

In a first aspect, the present invention provides a method for reducing a recipient's risk of developing platelet alloimmunization upon receiving transfused donor platelets by filtering the platelets through a leukoreduction filter; adding a photosensitizer to the platelets; irradiating the platelets and photosensitizer with light; transfusing the filter-leukoreduced irradiated platelets into a recipient; thereby reducing the risk of the recipient developing platelet alloimmunization upon receiving transfused donor platelets.

In an embodiment of this aspect, the leukoreduction filter can be a Pall PLF-1, Pall PL1-B, Fenwal PLS-5A filter. In another embodiment of this aspect, the photosensitizer is riboflavin. In a further embodiment of this aspect, the light is UV light at a wavelength of between 290-370 nm. In various embodiments of this aspect, the donor platelets can be from an antigenically mismatched donor, or else, the donor platelets can be from an antigenically matched donor.

In a second aspect, the present invention provides a method of preparing a toleragenic platelet composition that is substantially free or reduced of alloimmunizing cells by filtering the platelet composition to remove alloimmunizing cells; adding to the filtered platelet composition a photosensitizer comprising riboflavin; irradiating the filtered platelet composition and riboflavin with light at a wavelength of between 290-370 nm; and recovering the platelet composition as the toleragenic platelet composition.

In an embodiment of this aspect, the filtering is performed with a filter which can be a Pall PLF-1, Pall PL1-B, or Fenwal PLS-5A filter.

In a third aspect, the present invention provides a method of preventing platelet refractoriness in a recipient receiving platelets from an antigenically mismatched donor by filtering the platelets to be transfused through a leukoreduction filter; adding a photosensitizer to the platelets; irradiating the platelets and photosensitizer with light; and transfusing the irradiated platelets into the recipient; where the transfused platelets do not cause the recipient to develop platelet refractoriness, or delays or prevents the onset of platelet refractoriness.

In an embodiment of this aspect, the leukoreduction filter can be a Pall PLF-1, Pall PL1-B, or Fenwal PLS-5A filter. In another embodiment of this aspect, the photosensitizer is riboflavin. In a further embodiment of this aspect, the light is UV light at a wavelength of between 290-370 nm.

In a fourth aspect, the present invention provides a toleragenic platelet composition prepared by a process of filtering the platelets through a leukoreduction filter; adding a photosensitizer to the platelets; and irradiating the platelets and photosensitizer with light.

In an embodiment of this aspect, the leukoreduction filter can be a Pall PLF-1, Pall PL1-B, or Fenwal PLS-5A filter. In another embodiment of this aspect, the photosensitizer is riboflavin. In yet another embodiment of this aspect, the light is UV light at a wavelength of between 290-370 nm.

In a fifth aspect, the present invention provides a toleragenic platelet composition capable of not producing an immune reaction in a recipient receiving the platelet composition.

In an embodiment of this aspect, the toleragenic platelet composition, when administered to a recipient, delays the development of immunization to the platelet composition in the recipient.

### BRIEF DESCRIPTION OF THE DRAWINGS

FIG. 1 shows steps to modify the donor dog's platelets prior to transfusion.

FIG. 2 shows the gating strategy for characterization of cells remaining in PRP and after filtration.

FIGS. 3A-3D shows a characterization of cells remaining in platelet-rich-plasma (PRP) and after filtration with Fenwal PLS-5A or Pall PL-1B filters.

FIG. 4 shows a characterization of cells remaining after filtration with Fenwal PLS-5A or Pall PL-1B filters and centrifugation.

FIG. 5 shows time to alloimmune platelet refractoriness using single platelet modifications.

FIG. 6 shows time to alloimmune platelet refractoriness using combined platelet modifications.

### DETAILED DESCRIPTION

The present invention generally relates methods and compositions for the prevention or reduction of platelet alloimmunization and refractoriness using leukoreduction and light treatment regimes, such as those used in pathogen reduction processes.

It is to be understood that this invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural references unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

Whole blood collected from volunteer donors for transfusion into recipients is typically separated into components: red blood cells, white blood cells, platelets, and plasma, using apheresis, centrifugation procedures, or other known methods. Each of these separated blood components may be stored individually for later use and are used to treat a multiplicity of specific conditions and disease states. For example, the red blood cell component is used to treat anemia, the concentrated platelet component is used to prevent or control bleeding, and plasma is used frequently as a source of clotting factors for the treatment of congenital or acquired clotting factor deficiencies.

In cell separation procedures, there is usually some small percentage of other types of cells which are carried over into a separated blood component. When contaminating cells are carried over into a separated blood component in a high enough percentage to cause some undesired effect, the contaminating cells are considered to be undesirable. White blood cells, which may transmit infections such as HIV and CMV also cause other transfusion-related complications such as transfusion-associated Graft vs. Host Disease (TA-GVHD), alloimmunization and microchimerism.

Alloimmunization describes an immune response provoked in a transfused recipient by a donor alloantigen. Alloantigens include blood group substances (A, B, or AB) on erythrocytes and histocompatibility antigens expressed on white cells and platelets. An alloimmunizing cell as used herein is a cell which triggers an alloimmunization response against transfused platelets as described below.

Human Leukocyte Antigen (HLA) markers are found on the membranes of many different cell types, including white blood cells. HLA is the major histocompatibility complex (MHC) in humans, and contributes to the recognition of self v. non-self. Recognition by a transfusion recipient's immune system of differences in HLA antigens on the surface of transfused cells may be the first step in the rejection of transfused or transplanted tissues. Therefore, the phenomena of alloimmunization of recipients against HLA markers on donor blood is a major problem in transfusion medicine today. This issue arises in recipients of blood products due to the generation of antibodies against white blood cell HLA antigens in donor blood.

Platelets also express on their surface low levels of these HLA antigens. When a recipient of a whole blood or a blood component that contains donor white blood cells is transfused, the recipient may produce antibodies against the HLA antigens on the transfused donor's white blood cells. These antibodies may also lead to recognition and clearance of transfused platelets that carry this same marker. When this occurs, it becomes necessary to HLA match the platelet donor and recipient to assure that the recipient receiving the transfusion is able to maintain an adequate number of donor platelets in circulation. Finding an HLA-compatible donor is often a complicated, expensive and difficult procedure because of the complexity of the HLA system. Large numbers of potential platelet donors must be HLA-typed in order to have an available platelet donor registry that will contain compatible donors for most patients. In cases where recipients are very heavily transfused with blood or blood products from multiple donors and antibodies to many different HLA markers are generated, or where no suitable HLA-compatible platelet donor is available, death due to bleeding may occur.

One approach to preventing alloimmunization is to reduce the immunogenicity of the transfused blood products. As all transfused blood products are immunogenic and may eventually induce an immune response in most transfused recipients, any procedure that can prevent, reduce, or at least delay alloimmunization will be beneficial.

Since the immunization problem arises from the presence of white cells in the donated blood products, the elimination of white cells from these products would be expected to reduce the alloimmunization rates. Gamma irradiation of blood products, which kills the cells but does not remove them from the blood product to be transfused, has not been shown to be able to prevent alloimmunization. It is likely that this is due to the fact that the irradiated white cells are still present and capable of presenting antigens to the recipient's immune system. This hypothesis is supported by studies that have shown that gamma-irradiated lymphocytes are still able to stimulate other donor's lymphocytes in mixed lymphocyte cultures.

Filtration of white blood cells from blood or blood products to be transfused has been shown to be capable of reducing alloimmunization rates. This has been demonstrated based on an extensive clinical study known as the TRAP Trial. The TRAP Trial was conducted as a multi-institutional study between 1995 and 1997 and results were subsequently published in the NEJM in 1997 (Trial to Reduce Alloimmunization to Platelets Study Group. Leukocyte reduction and ultraviolet B irradiation of platelets to prevent alloimmunization and refractoriness to platelet transfusions. N Engl J Med. 1997; 337:1861-1869). The data from that study suggested that leukoreduction significantly decreased the likelihood of alloimmunization in patients from 45% for non-leukoreduced, untreated products to 17% to 18% for filter leukoreduced products. The remaining levels of alloimmunization that were observed in the TRAP Trial were believed to be due to residual white blood cells that were not removed by filtration. As a result of this work, platelet products have been filtered or centrifuged by a variety of methods to remove white blood cells. However, even the best white blood cell filters or centrifuge leukoreduction methods cannot remove 100% of the white blood cells, and those left behind are potentially able to stimulate antibody production against the HLA markers on the remaining cells. A decrease in the alloimmunization rate from 45% of patients receiving standard platelets to 17% to 18% is significant, but still leaves several tens of thousands of cases of alloimmunization occurring on an annual basis. Furthermore, when a subset analysis was done of the 36 patients in the TRAP Trial who had never had prior antigen exposure from transfusion or pregnancy and who received all of their transfusions as leukoreduced, the immunization rate was still 19%. The patients in the TRAP Trial all had Acute Myelogenous Leukemia and were undergoing potentially immunosuppressive induction chemotherapy. Thus, it is likely that the residual alloimmunization rates would have been much higher in an immunocompetent patient population.

In the same TRAP study, treatment of platelet products with ultraviolet B (UVB) light was also evaluated. In the case of UVB treatment, the results were equivalent to those obtained with filtration leukoreduction. The work was consistent with prior studies that showed that UVB treated platelet products possessed significantly reduced alloimmunization responses (Blundell et al. Transfusion 1996; 36: 296-302). This was believed to be due to changes in white cells induced by UVB that cause them to present their antigens and have those antigens processed differently from non-irradiated cells by the patient's immune system. The result is that antibody generation is significantly suppressed for UVB treated products. Although the results were positive, the UVB treatment described in the TRAP study was never implemented.

Photosensitizers, or compounds which absorb light of a defined wavelength and transfer the absorbed energy to an electron acceptor may be a solution to some of the above problems. Instead of physically removing contaminating white blood cells as leukoreduction procedures do, photosensitizers chemically inactivate the undesirable white cells without substantially damaging the desirable components of blood.

There are many photosensitizer compounds known in the art to be useful for inactivating undesirable cells and/or other infectious particles. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, anthroquinones and endogenous photosensitizers.

When illuminated with UV light, riboflavin, or 7,8-dimethyl-10 ribityl isoalloxazine, an endogenous photosensitizer, has been shown to help reduce transfusion-related complications in a blood transfusion recipient. This is taught in U.S. Pat. No. 7,648,699.

In those instances where filtration of blood or a blood component to be transfused into a recipient does not remove enough of the white blood cells to prevent alloimmunity, we have discovered that adding one or more additional treatments to inactivate the remaining white blood cells is surprisingly effective. Additional treatments may include the addition of a photosensitizer to the filter leukoreduced blood/blood component. The photosensitizer and filter leukoreduced blood/blood component may then be exposed to light for a sufficient amount of time to reduce the immunogenicity of the remaining white blood cells in the donor blood to such an extent that little or no immune response to the donor blood is generated by the recipient.

Any of a number of leukoreduction methods known in the art may be used in the practice of the present invention. Leukoreduction refers generally to any process which physically removes immunogenic cells, particularly, white blood cells (or leukocytes), from the blood or blood components supplied for blood transfusion. After the removal of the immunogenic cells or leukocytes, the blood product is said to be leukoreduced. Known methods for performing leukoreduction include, but are not limited, to centrifugation and filtration. In performing centrifugation to produce leukoreduced platelets, differential centrifugation is performed to separate platelets from immunogenic cells such as WBCs, as known in the art. Centrifugation may result in the leukoreduction of a sample by at least 5%, 10%, 20%, 30%, 40%, 50%, 60%, 70%, 80%, 90%, or 100%, and all numbers in between, as compared to non-leukoreduced samples.

A leukoreduction filter is any filter which is capable of physically removing immunogenic cells, particularly, white blood cells (or leukocytes), from the blood or blood components supplied for blood transfusion using filtration methods. Leukoreduction filters are known in the art and are commercially available. Examples of leukoreduction filters include, but are not limited, to those made by Fenwal Blood Technologies (e.g., PLS-5A filter), Pall Corporation (e.g., Pall PLF-1, PL-1B, LeukoGuard RS, Leukotrap SC PL, LRF-10, Purecell LRF, PXL 8 and 12, PXLA, RCXL 1 and 2), among others. Filtration may result in the leukoreduction of a sample by at least 5%, 10%, 20%, 30%, 40%, 50%, 60%, 70%, 80%, 90%, or 100%, and all numbers in between, as compared to untreated samples.

Any of a number of light or irradiation treatment methods known in the art may be used in the practice of the present invention. The light source may be of many wavelengths, with wavelengths in the UV range being advantageous. Light treatment or irradiation can be performed with or without a photosensitizer as described below. In some aspects of the invention, the light treatment regime is one which is used in pathogen reduction processes using light or irradiation. Such pathogen reduction processes typically employ irradiation in the presence or absence of a photosensitizer to cross-link pathogenic cellular components such as nucleic acids.

Among the pathogen reduction methods that may be used in the practice of the present invention include, without limitation, those that rely on riboflavin and UV light (e.g., Mirasol Pathogen Reduction Technologies System from CardianBCT, Lakewood, Colo.); those that rely on psoralen and UV light (e.g., Cerus INTERCEPT Blood System, Concord, Calif.); and those that rely solely on UV-C light treatment (e.g., Seltsam and Muller, Transfus Med Hemother, 2011, 38: 43-54; Mohr et al., Transfusion, 2009, 49: 2612-24).

Photosensitizers useful in the present invention include endogenous photosensitizers. The term “endogenous” means naturally found in a human or mammalian body, either as a result of synthesis by the body or because of ingestion as an essential foodstuff (e.g., vitamins) or formation of metabolites and/or byproducts in vivo. When endogenous photosensitizers are used, particularly when such photosensitizers are not inherently toxic or do not yield toxic photoproducts after photoradiation, no removal or purification step is required after decontamination, and the decontaminated product can be directly administered to a recipient in need of its therapeutic effect.

Examples of such endogenous photosensitizers which may be used in this invention are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavin adenine dinucleotide [FAD]) and alloxazine mononucleotide (also known as flavin mononucleotide [FMN] and riboflavine-5-phosphate). The term “alloxazine” includes isoalloxazines.

Use of endogenous isoalloxazines as a photosensitizer to pathogen reduce blood and blood components are described in U.S. Pat. Nos. 6,258,577 and 6,277,337 both issued to Goodrich et al.

Generally, whole blood is withdrawn from a donor and separated into components such as platelets, plasma and red blood cells, either manually by centrifugation procedures, or automatically. If separated automatically, such as by apheresis, an apheresis machine such as a Trima apheresis machine (CaridianBCT, Inc., Lakewood, Colo.) can be used, or a whole blood separation machine such as an Atreus whole blood separation machine (CaridianBCT Inc., Lakewood, Colo.) can be used.

The non-immunogenic and toleragenic platelet compositions produced as a result of both filtration or centrifuge leukoreduction and irradiation of riboflavin with UV light may be used for tolerance induction. Toleragenic refers to the capacity of a composition to not generate an immunologic response to a given antigen that, under normal circumstances would likely induce cell-mediated or humoral immunity. An immunogenic reaction generally occurs at the earliest 10-14 days after platelet transfusion in a naive recipient. Thus, a toleragenic platelet composition is one which does not produce an immunogenic reaction more than 10-14 days after platelet transfusion, preferably more than 3 weeks, more than 4 weeks, more than 5 weeks, more than 6 weeks, more than 7 weeks, or more than 8 or greater weeks after platelet transfusion. Tolerance is induced by administering transfusions, generally repeated transfusions, of the treated platelet composition to a recipient.

Platelet refractoriness occurs when a recipient fails to obtain a satisfactory response to two or more successive platelet transfusions. In clinical practice, there is usually little doubt when patients are failing to have satisfactory responses to a platelet transfusion, as indicated by no increase in platelet count on the day of or the day after a platelet transfusion.

To determine whether platelet refractoriness has occurred as a result of alloimmunity, platelet responses are measured in conjunction with antibody assays using donor lymphocytes or platelets as the target cell. Platelet responses are measured by determining pre-and post-transfusion platelet counts and calculating platelet increments, % platelet recovery, or corrected count increments. A recipient is considered platelet alloimmune refractory to the donor's platelets if the one-hour post-transfusion Corrected Count Increment, CCI is 7,500 (namely, 0-7,500 and all numbers in between) or the 24-hour post-transfusion CCI is 4,500 (namely, 0-4,500 and all numbers in between), along with a positive antibody assay against the donor's lymphocytes or platelets.

The following examples of specific aspects for carrying out the present invention are offered for illustrative purposes only, and are not intended to limit the scope of the present invention in any way.

### EXAMPLES

### Example 1: Experimental Design and Methods

The following experimental methods are used in the Examples that follow.

### Experimental Design of the Dog Platelet Transfusion Studies

1) Perform baseline autologous radiolabeled platelet recovery and survival measurements in recipient dogs to ensure that their data is normal.

2) Select DLA, DRB mismatched and crossmatch negative random donor/recipient pairs.

3) Prepare platelets weekly from a single random donor.

4) Donor dog's platelets are unmodified (standard), filter-leukoreduced, γ-irradiated, UV-irradiated plus riboflavin (Mirasol pathogen reduction technology), or treatments are combined.

5) Donor dog's platelets, after modification, are radiochromium labeled prior to recipient transfusion.

6) Serial blood samples are drawn from the recipient to determine recovery and survival of the donor dog's radiolabeled platelets.

7) Recipient receives up to 8 weekly transfusions from their donor or until they become platelet refractory.

8) Primary Endpoint: Refractoriness is defined as <5% of the radiolabeled donor dog's platelets still circulating in the recipient at 24 hours post-transfusion after two sequential transfusions.

9) Autologous radiolabeled platelet recovery and survival measurements in the recipient dogs are repeated after donor platelet transfusions are completed to ensure that any refractoriness to the donor dog's platelets is due to alloimmunization rather than a change in the condition of the recipient dog that would not allow even autologous platelets to circulate normally.

### Modification Of The Donor's Platelets

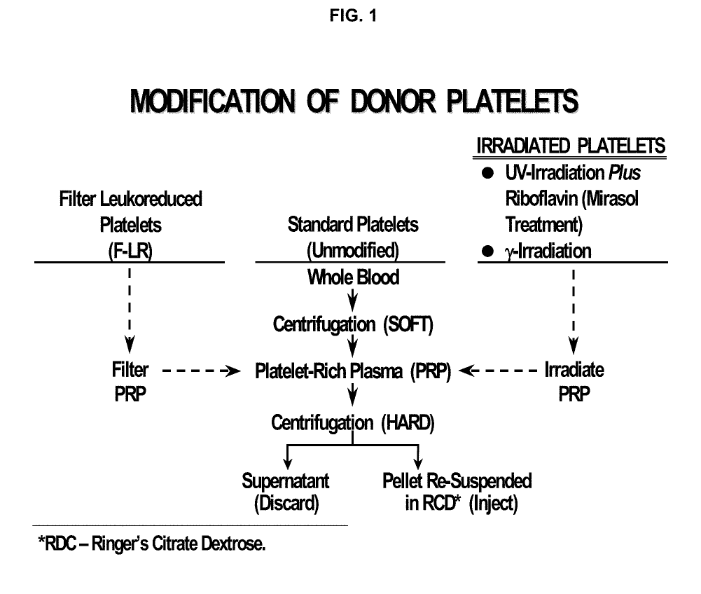

The steps used to modify a donor dog's platelets prior to transfusion are shown in FIG. 1.

### Lymphocyte Antigen (DLA) Typing

Nucleotide sequence alignments for approximately 50 DLA-DRB alleles were available online. Since most sequence variations are located in second exons of class II genes, the amplification primer sequences for various DRB loci and alleles were selected from the conserved regions of 5′ and 3′ ends of exon 2. Oligo-nucleotide probes were selected from regions with sequence variation, and probes were designed to ensure uniform melting temperatures (T_{M}) and to enable uniform hybridization and wash conditions. The oligoprobes were poly(T) tailed and bound on nylon membranes (oligoblot). Following specific amplification, the individual amplicons were hybridized to a single oligoblot containing multiple probes defining various DLA-DRB alleles. Excess of unhybridized PCR products were removed in stringent washes, and the oligoblots were subjected to an immunological detection step. Positive reactions were visualized either as color precipitate or on X-ray film depending on the method of preference.

### Platelet And WBC Antibody Testing

Antibody identification studies were performed baseline and on weekly blood samples drawn from the recipient dogs to detect IgM and IgG antibodies to donor platelets and WBCs. Serum samples were also tested against the recipients' autologous platelets and WBCs as negative controls. Antisera from alloimmune platelet refractory animals were pooled and run as a positive control against both autologous and donor platelets and WBCs.

A flow cytometric assay was used to detect anti-IgG or anti-IgM antibodies to donor platelets, B cells, and CD8 positive white cells. Platelets and WBCs were isolated from donor and recipient's whole blood, and these cells were added to a tissue culture plate. Platelets were adjusted to 300,000/well and WBCs to 35,000/well. Dog sera were added to the wells along with cell identification reagents, followed by FITC-labeled anti-dog IgG and IgM reagents. Cells were incubated with the reagents, washed, staining buffer was added, and mean fluorescence of platelets and lymphocytes were detected using the FACScan. Results were considered positive for recipient antibodies against the donor's platelets or WBCs if the test sera were 1.3 times the donor's autologous control sera tested with the same cells.

### WBC Identification

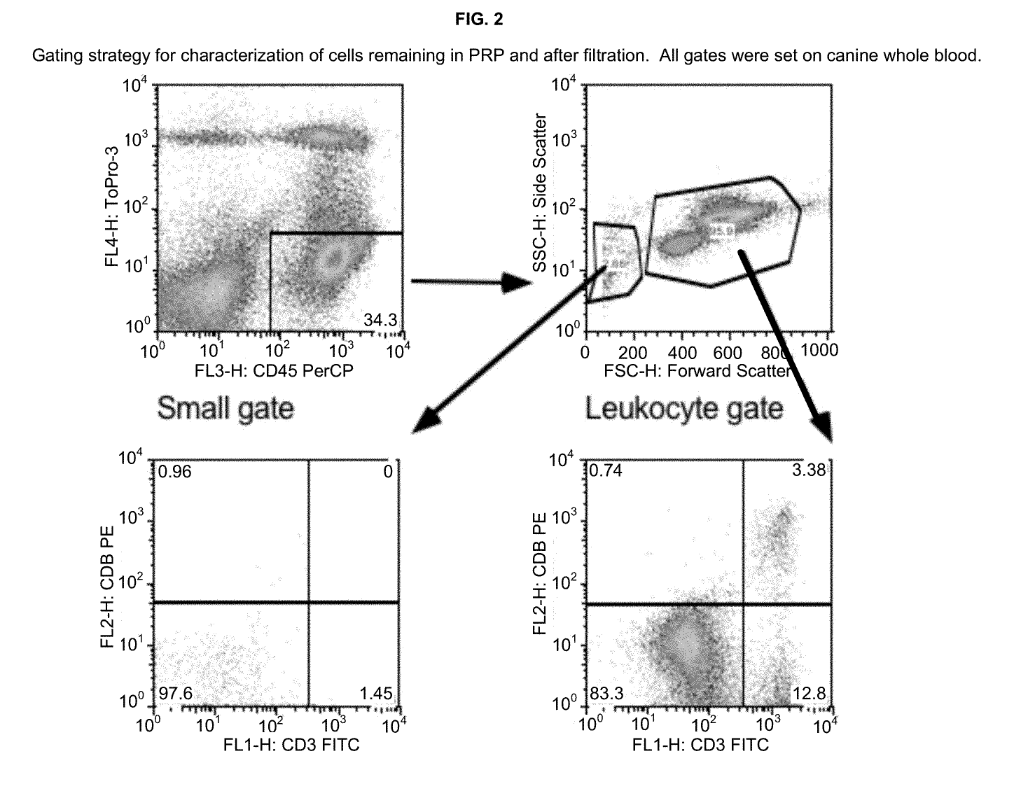

WBC identification was performed using a panel of anti-canine antibodies and a BD Facscalibur flow cytometer to detect the cell types and CD45 positive microparticles after filtration as compared to whole blood preparations. Briefly, the blood was processed and filtered using the same method used for transfusions. A whole blood sample was used as a reference sample, a platelet-rich-plasma (PRP) sample as another reference, and then the processed and filtered PRP samples were analyzed. The panel used to detect CD45 positive and topro negative (live) cells and microparticles was as follows: DM5 (granulocytes), B cell, class II, CD4, CD14, CD34, CD3, CD8, and isotype (to rule out nonspecific binding). In this way, we could evaluate any differences detected by this panel between the filters studied. The PRP was passed through a Fenwal PLS-5A or Pall PL-1B filter according to the method described above (FIG. 1). The filtrate was analyzed for the percentage and number of leukocytes that remained (FIG. 2).

### Example 2: Effectiveness of Different Leukoreduction Methods

This example provides methods of modifying a donor dog's platelets prior to transfusion in a dog platelet transfusion model that would prevent alloimmune platelet refractoriness. We have previously demonstrated that methods of preventing platelet alloimmunization in a dog model could be successfully transferred to patients. Specifically, we have demonstrated that UV-B irradiation that was 45% successful in preventing alloimmunization in the dog was 81% successful in patients in the largest prevention of platelet alloimmunization trial ever conducted in patients (TRAP Trial). (See Slichter S J, Deeg H J, Kennedy M S. Prevention of platelet alloimmunization in dogs with systemic cyclosporine and by UV-irradiation or cyclosporine-loading of donor platelets. Blood 1987; 69(2):414-418; The Trial To Reduce Alloimmunization To Platelets Study Group. Leukocyte reduction and ultraviolet B irradiation of platelets to prevent alloimmunization and refractoriness to platelet transfusions. N Engl J Med 1997; 337:1861-1869.) The fact that UV-B irradiation was even more successful in patients than in the dog is probably because the dogs had a normal immune system while being transfused versus a compromised immune system in the study patients who were receiving induction chemotherapy for acute myelogenous leukemia (AML). Therefore, any beneficial approach in the dog is likely to be even more successful in cancer patients receiving chemotherapy or stem cell transplants. These patients, who often receive prolonged platelet therapy, would benefit the most from methods to prevent alloimmunization.

We have done prior studies in our dog model suggesting that just a quantitative reduction in the number of residual white blood cells (WBCs) was not sufficient to prevent alloimmunization. It is known that transfused WBCs contain antigen presenting cells (APCs) that present donor antigens to the recipient's immune system leading to alloimmunization. In fact, different methods of leukoreduction using centrifugation (C-LR) versus filtration (F-LR) that both produce the same levels of leukoreduction from 10⁶ WBCs/transfusion without leukoreduction to 10⁴ WBCs/transfusion with either method of leukoreduction produce different transfusion outcomes (Table 1). Even different filters produced different results.

TABLE 1
EFFECTS OF DIFFERENT METHODS OF
LEUKOREDUCTION ON ACCEPTANCE
OF DONOR PLATELETS

| | ACCEPTANCE | |
|---|---|---|
| | RATES | |
| | # Donor's Accepted*/ | |
| Platelet Modificaion | # Recipients (%) | |
| None (Standard) | 1/7 | (14%) |
| Single Modification: | | |
| Centrifuge Leukoreduction (C-LR) | 3/21 | (14%) |
| Filter Leukoreductiond (F-LR) | | |
| Pall PLF-1 Filter | 4/13 | (31%) |
| Pall PL1-B Filter | 2/7 | (29%) |
| Fenwal PLS-5A | 4/6 | (66%) |

*Donor platelets accepted for 8 weeks.

As can be seen from Table 1, varying degrees of acceptance are obtained when different methods of leukoreduction are used, ranging from centrifuge leukoreduction (C-LR), which when used alone gives no better rate of acceptance than no treatment (14%). The Fenwal PLS-5A filter gave the highest rate of acceptance at 66%.

We then assessed whether combining different methods of preventing alloimmunization would increase donor platelet acceptance rates. Combining UV-B irradiation with C-LR gave acceptance rates of 55% compared to acceptance rates of 71% when UV-B was combined with F-LR. Based on acceptance rates for a single modification (C-R=14%, Pall PLF-1 F-LR=31%, and UV-B irradiation=45%), combinations of LR with UV-B irradiation were additive in the results achieved (Table 2).

TABLE 2
DONOR PLATELETS

| | # Donor's Accepted*/ | |
|---|---|---|
| | # Recipients (%) | |
| C-LR plus UV-B | 6/11 | (55%) |
| F-LR** plus UV-B | 10/14 | (71%) |

*Donors accepted for 8 weeks.
**Pall PLF-1 filter.

Surprisingly, when C-LR was combined with two of the filters tested (Pall's PLF-1 and Fenwal's PLS-5A), donor acceptance rates were 95% to 100% versus only 50% with Pall's PL1-B filter (Table 3). The results with the first two filters (Pall's PLF-1 and Fenwal's PLS-5A) were synergistic rather than additive as obtained with the last filter.

TABLE 3

| | EFFECTS OF COMBINING F-LR WITH C-LR ON | |
|---|---|---|
| | ACCEPTANCE OF DONOR PLATELETS | |
| | F-LR | F-LR/C-LR* |
| | # Donors Accepted**/ | # Donors Accepted**/ |
| Filter | # Recipients (%) | # Recipients (%) |

| | | | |
|---|---|---|---|
| Pall | | | |
| PLF-1 PL1B Fenwal PLS-5A | 4/13 (31%) 2/7 (29%) 4/6 (66%) | 18/19 (95%) 6/12 (50%) 10/10 (100%) | 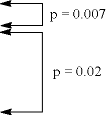 |

*Residual WBCs all <3 × 10³ (Lower limit of detection of the assay).
**Donor platelets accepted for 8 weeks.

The results shown in Table 3 are consistent with the idea that F-LR with Pall's PLF-1 and Fenwal's PLS-5A must remove different types of WBCs than does C-LR. Therefore, combining F-LR with C-LR gives almost complete prevention of alloimmune platelet refractoriness. Furthermore, although both the PLF-1 and PL1-B filters were made by Pall and they produced the same amount of leukoreduction, they must be removing and/or leaving different types of WBCs because C-LR does not produce the same results when combined with the PLF-1 filter (95% donor acceptance rates) versus the PL1-B filter (50% donor acceptance rates).

We then assessed whether we could improve the acceptance rates of the PL1-B filter by combining F-LR/C-LR with either UV-B irradiation or y-irradiation (Table 4). Unexpectedly, adding UV-B irradiation to F-LR/C-LR did not improve the results while adding y-irradiation was 100% successful.

TABLE 4
ADDITIONAL MODIFICATIONS OF PL1-B FILTERED,
CENTRIFUGED LEUKOREDUCED PLATELETS

| | ACCEPTANCE RATES | |
|---|---|---|
| | # Donors Accepted*/ | |
| Platelet Modification | # Recipients (%) | |
| PL1-B (F-LR/C-LR) | 6/12 | (50%) |
| PL1-B (F-LR/C-LR/UV-B irradiated) | 3/8 | (38%) |
| PL1-B (F-LR/C-LR/γ-irradiated) | 7/7 | (100%) |

*Donor platelets accepted for 8 weeks.

### Example 3: WBC Identification

This Example describes experiments designed to characterize the WBCs that are removed and those that remain after F-LR and combined F-LR/C-LR procedures using monoclonal antibodies specific for canine WBCs.

The WBC identification method using FACS as described in Example 1 was employed for these studies.

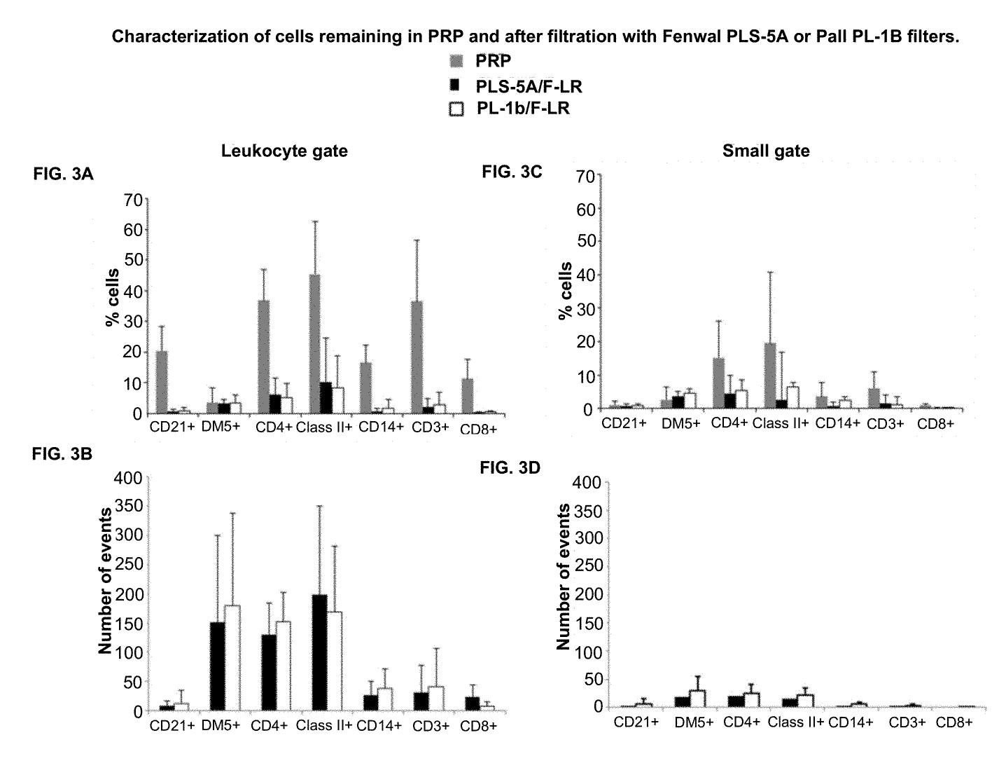

As expected, the PRP was enriched for lymphocytes for example, B cells (CD21⁺), T cells (CD3⁺) and DLA Class II positive (C11+) cells (FIG. 3A). As canine granulocytes, monocytes and T cells express CD4; in our analysis CD4 positive cells are a mixture of all these cell types. Both Fenwal PLS-5A and Pall PL-1B filters removed most of the lymphocytes with the percentage of CD8⁺ T cells and B cells in the leukocyte gate below 1% of the total CD45+ cells (FIG. 3A). The analysis of the number of events showed that most of the cells remaining were granulocytes (DM5⁺cells) and CII+ cells (FIG. 3B). We also evaluated the percentage of cells and number of events in the low forward and size scatter gate (small gate; see FIG. 2). This gate could consist of fragments of cells generated by the filters or microparticles. As in the leukocyte gate, the remaining cells were mostly CII+ cells and granulocytes (FIGS. 3C, 3D). Further studies are needed to verify the properties of cells/particles within the small gate.

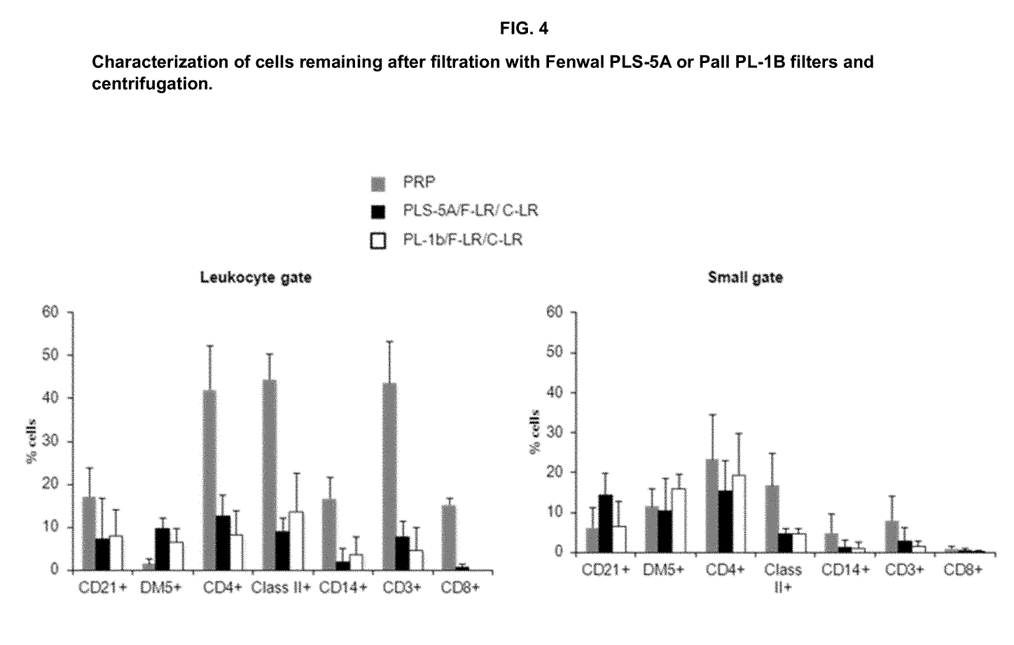

To investigate whether combined F-LR/C-LR removed and/or left different types of WBCs, we analyzed cells left in the supernatant after filtration and low speed centrifugation. Surprisingly, after filtration followed by centrifugation (F-LR/C-LR), the remaining populations of cells were similar to F-LR alone except there was a slight enrichment of CD21⁺ cells in the small gate (FIG. 4). This difference did not reach statistical significance.

Our results indicate that both Fenwal PLS-5A and Pall PL-1B filters remove 90% of leukocytes from PRP. The residual cells and fragments and/or microparticles after F-LR are mostly granulocytes and CII+ cells. We could detect no difference in remaining cell populations between F-LR and F-LR/C-LR.

### Example 4: Modified Platelet Transfusion Experiments To Prevent Platelet Alloimmunization

Following the results we obtained with y-irradiation, combined with PL1-B F-LR/C-LR in our prior studies (Example 2; Table 4), we proceeded to determine the effects of γ-irradiation alone or when combined with F-LR. F-LR and γ-irradiation are both processes that are routinely performed by blood centers.

In Table 5, we first evaluated whether y-irradiation alone could prevent alloimmune platelet refractoriness.

TABLE 5
SINGLE PLATELET MODIFICATIONS

| | # Donors Accepted*/ | |
|---|---|---|
| | # Recipients(%) | |
| None (Standard) | 1/7 | (14%) |
| Filtration: | | |
| Pall PL-1B Filter | 2/7 | (29%) |
| Fenwal PLS-5A Filter | 4/6 | (66%) |
| γ-irradiation | 0/5 | (0%) |
| Mirasol Treatment | 1/7 | (14%) |

*Donor transfusions accepted for 8 weeks.

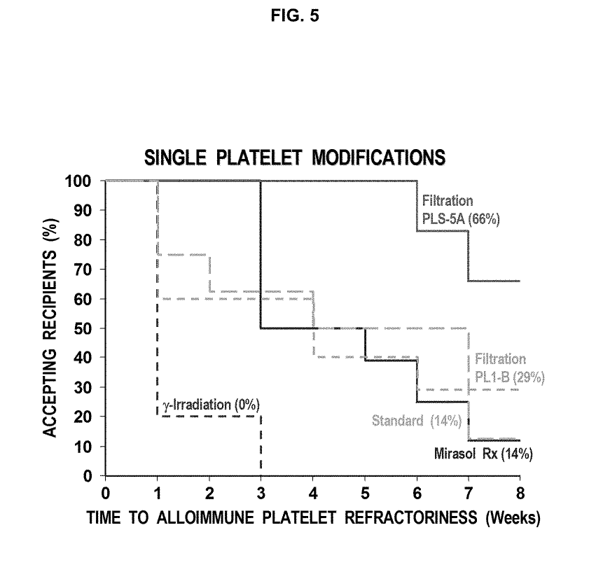

None of the 5 dogs given y-irradiated donor platelets accepted these platelets, and the time to develop platelet refractoriness was even shorter than the time required for recipients to become refractory to standard (unmodified) donor platelets (FIG. 5).

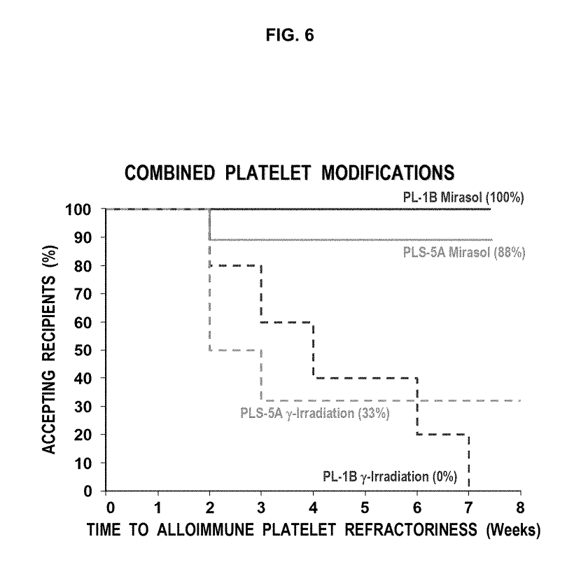

We next determined whether y-irradiation combined with F-LR would improve the results achieved with F-LR alone. Adding y-irradiation not only did not improve the acceptance of F-LR donor platelets (Table 6) or time to platelet refractoriness (FIG. 6), γ-irradiation may actually have reduced the effectiveness of F-LR using both the Pall PL1-B and Fenwal PLS-5A filters.

TABLE 6
COMBINED PLATELET MODIFICATIONS:
FILTER LEUKOREDUCTION PLUS γ-IRRADIATION OR
MIRASOL TREATMENT

| | | | MIRASOL | |
|---|---|---|---|---|
| | γ-IRRADIATION | | TREATMENT | |
| | # Donors Accepted*/ | | # Donors Accepted*/ | |
| Filtration | # Recipients (%) | | # Recipients (%) | |
| Pall PL1-B** | 0/5 | (0%) | 7/7 | (100%) |
| Fenwal PLS-5A*** | 2/6 | (33%) | 7/8 | (88%) |
| Total | 2/11 | (18%) | 14/15 | (93%) |

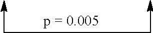

*Donor transfusions accepted for 8 weeks.
**PL1-B Only - 2/7 (29%).
***PLS-5A Only - 4/6 (66%).

### Example 5: Combination of UV Light Treatment and Filtration

This Example provides experiments to determine whether a pathogen-reduction technology could prevent alloimmune platelet refractoriness in our dog model. The technology involves adding riboflavin to the platelets followed by UV-irradiation (Mirasol treatment). This process prevents replication of DNA and RNA in bacteria, viruses, and WBCs suggesting that it might prevent alloimmunization due to contaminating WBCs in the transfused platelets.

Treating the donor's platelets with Mirasol did not prevent platelet refractoriness, and the percentage of accepting recipients and time to refractoriness was the same as standard (unmodified) platelet transfusions (Table 5 and FIG. 5). In contrast, when Mirasol treatment was combined with either the PL1-B or PLS-5A filters, acceptance rates were 100% and 88%, respectively (Table 6 and FIG. 6). Using y-irradiation combined with F-LR was effective at preventing alloimmune platelet refractoriness in only 2/11 (18%) of dogs compared to 14/15 (93%) of dogs who received Mirasol treated plus F-LR platelets (p=0.005).

In these experiments, antibody results correlated with platelet transfusion results 65% of the time for antibody tests against platelets and 67% of the time for antibody tests against WBCs; i.e., the assays were positive when the recipient developed refractoriness to donor platelets or antibodies were not detected when donor platelets were accepted for 8 weeks. The biggest problem with the antibody assays was failure to detect antibodies when the dog was platelet refractory; i.e., 30% of the recipients were negative for both platelet and WBC antibodies when the dog was platelet refractory. In contrast, antibodies were detected in only 5% and 2% of platelet and WBC antibody tests when the recipient did not become refractory to donor platelets. The failure to detect antibodies in 30% of the recipients who were refractory to donor platelets emphasizes the relevance of using refractoriness to donor platelets as the primary endpoint of our studies. Clinicians are much more interested in how well patients respond to platelet transfusions rather than their antibody status. As autologous radiolabeled platelet recovery and survival measurements at the end of the donor platelet transfusion experiments were all unchanged from baseline values, we are confident that refractoriness to donor platelets in our studies was secondary to alloimmunization, even in the absence of positive antibody tests.

While specific aspects of the invention have been described and illustrated, such aspects should be considered illustrative of the invention only and not as limiting the invention as construed in accordance with the accompanying claims.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1-18. (canceled)

19. A method for reducing a recipient's risk of developing platelet alloimmunization upon receiving transfused donor platelets comprising:
obtaining platelet-rich plasma (PRP) from one or more donors;
filtering the PRP through a leukoreduction filter that yields a filter-leukoreduced platelet composition comprising less than 1% CD8+ T cells;
adding riboflavin to the filter-leukoreduced platelet composition;
irradiating the filter-leukoreduced platelet composition and riboflavin with UV light at a wavelength of between 290-370 nm; and
transfusing the filter-leukoreduced irradiated platelet composition into a recipient;
thereby reducing the recipient's risk of developing platelet alloimmunization upon receiving transfused donor platelets.

20. The method of claim 19, wherein the donor platelets are from an antigenically matched donor.

21. The method of claim 19, wherein the donor platelets are from an antigenically mismatched donor.

22. The method of claim 19, wherein the transfused filter-leukoreduced irradiated platelet composition has an acceptance rate of 88% or greater.

23. The method of claim 19, wherein the filter-leukoreduced irradiated platelet composition comprises a leukocyte population comprising less than 1% B cells as compared to a PRP before filtering.

24. The method of claim 19, wherein the transfused filter-leukoreduced irradiated platelet composition is accepted by the recipient for 8 weeks or more.

25. A method of reducing or preventing platelet refractoriness in a recipient receiving platelets from an antigenically mismatched donor comprising:
obtaining platelet-rich plasma (PRP) from a donor;
filtering the PRP through a leukoreduction filter that yields a filter-leukoreduced platelet composition comprising less than 1% CD8+ T cells;
adding riboflavin to the filter-leukoreduced platelet composition;
irradiating the filter-leukoreduced platelet composition and riboflavin with UV light at a wavelength of between 290-370 nm; and
transfusing the filter-leukoreduced irradiated platelet composition into a recipient;
thereby reducing or preventing platelet refractoriness in a recipient receiving platelets from an antigenically mismatched donor.

26. The method of claim 25, wherein the transfused filter-leukoreduced irradiated platelet composition has an acceptance rate of 88% or greater.

27. The method of claim 25, wherein the filter-leukoreduced irradiated platelet composition comprises a leukocyte population comprising less than 1% B cells as compared to a PRP before filtering.

28. The method of claim 25, wherein the transfused filter-leukoreduced irradiated platelet composition is accepted by the recipient for 8 weeks or more.

29. The method of claim 25, wherein platelet refractoriness occurs when <5% of the donor platelets circulate in the recipient at 24 hours post-transfusion after two sequential transfusions.

30. The method of claim 25, further comprising measuring platelet refractoriness by determining pre-and post-transfusion platelet counts and calculating platelet increments, % platelet recovery, or corrected count increments.
